(19) United States
(12) Patent Application Publication
(10) Pub. No.: US 2025/0022261 A1
(43) Pub. Date: Jan. 16, 2025

(54) APPARATUS AND METHOD FOR AUDITING OF ARTIFICIAL INTELLIGENCE-BASED MEDICAL IMAGE SEGMENTATION

(71) Applicant: CLARIPI INC., Seoul (KR)

(72) Inventors: Si Hwan KIM, Seoul (KR); Jong Hyo KIM, Seoul (KR)

(21) Appl. No.: 18/771,443

(22) Filed: Jul. 12, 2024

(30) Foreign Application Priority Data
Jul. 13, 2023 (KR) 10-2023-0091368

(51) Int. Cl. G06V 10/776; G06V 10/26; G06V 10/774; G06V 20/70

(52) U.S. Cl. CPC G06V 10/776; G06V 10/267; G06V 10/774; G06V 20/70; G06V 2201/03

(57) ABSTRACT

Disclosed is a method of auditing of artificial intelligence-based medical image segmentation, including: performing preprocessing to generate a preprocessed segmentation image by receiving an input medical image and an output segmentation image provided from a medical image segmentation device and preprocessing the output segmentation image based on the input medical image; generating a heatmap image to generate a segmentation error heatmap image, which includes a segmentation error region in the preprocessed segmentation image, by inputting the preprocessed segmentation image to a deep learning model trained in advance; calculating an error risk to calculate a segmentation error risk for the segmentation error region based on pixel values of the segmentation error heatmap image; and providing auditing information to provide the auditing information for auditing accuracy of the output segmentation image based on the calculated segmentation error risk to a user.

### CROSS REFERENCE TO RELATED APPLICATION

Priority to Korean patent application number 10-2023-0091368 filed on Jul. 13, 2023 the entire disclosure of which is incorporated by reference herein, is claimed.

### BACKGROUND OF THE INVENTION

### Field of the Invention

The disclosure relates to an apparatus and method for auditing of an artificial intelligence-based medical image segmentation device, and more particularly to an apparatus and method for auditing of artificial intelligence-based medical image segmentation device, which compares an input image input to a medical image segmentation device and a segmentation result output from the medical image segmentation device.

### Description of the Related Art

In general, an X-ray, a computed tomography (CT), a magnetic resonance imaging (MRI), and the like medical apparatuses are used to acquire medical images. In modern medicine, the medical images acquired through such medical apparatuses are used as a very important basis for the presence and characteristics of lesions to make decisions in a process of diagnosing and treating a patient.

With the advancement of artificial intelligence technologies, various technologies for processing medical images based on artificial intelligence have been researched and developed. For example, medical image segmentation refers to a technology that separates a region of interest required for a lesion diagnosis from 3D medical image data. Through the medical image segmentation, the regions of interest segmented from an entire image are visualized as 2D or 3D images, thereby improving the accuracy and efficiency of the lesion diagnosis. Thus, various attempts have been made to apply the artificial intelligence technology to the medical image segmentation.

For example, a related art to artificial intelligence-based medical image processing has been disclosed in Korean Patent No. 10-1760287 (titled “DEVICE AND METHOD FOR MEDICAL IMAGE SEGMENTATION,” and registered on Jul. 17, 2017). The related art is characterized in that the regions of interest are segmented from medical images based on the artificial intelligence.

Most researches and developments of the artificial intelligence-based image segmentation technologies have been conducted only to segment the regions of interest from the medical images. However, to ensure the stable operations of the image segmentation technology, it is necessary to determine whether results output from a medical image segmentation device are accurate. Accordingly, a technology for auditing the output of accurate segmentation results in an image segmentation process is required.

### SUMMARY OF THE INVENTION

An aspect of the disclosure is to provide an apparatus and method for auditing artificial intelligence-based medical image segmentation, which audits whether results output in an image segmentation processing process are accurate.

According to an embodiment of the disclosure, A method of auditing of artificial intelligence-based medical image segmentation includes: performing preprocessing to generate a preprocessed segmentation image by receiving an input medical image and an output segmentation image provided from a medical image segmentation device and preprocessing the output segmentation image based on the input medical image; generating a heatmap image to generate a segmentation error heatmap image, which includes a segmentation error region in the preprocessed segmentation image, by inputting the preprocessed segmentation image to a deep learning model trained in advance; calculating an error risk to calculate a segmentation error risk for the segmentation error region based on pixel values of the segmentation error heatmap image; and providing auditing information to provide the auditing information for auditing accuracy of the output segmentation image based on the calculated segmentation error risk to a user.

The performance of the preprocessing may include: receiving the input medical image having a first data format and the output segmentation image having a second data format provided from the medical image segmentation device; and converting the output segmentation image having the second data format to have the same data format as the first data format of the input medical image.

The performance of the preprocessing may include: receiving the input medical image and the output segmentation image, in which a segment region of the input medical image is subjected to a color overlay, provided from the medical image segmentation device; separating the output segmentation image into color components and gray components; and converting an image corresponding to the color components to have the same format as a format of the input medical image.

The generation of the heatmap image may include: receiving the input medical image and the preprocessed segmentation image; and preprocessed applying the segmentation image to the deep learning model, which distinguishes between a normal image and an abnormal image, to output the segmentation error region in a heatmap format.

The method may further include: in training the deep learning model, generating a plurality of output segmentation images by inputting the plurality of input medical images to the medical image segmentation device, labeling the plurality of output segmentation images divisionally with normal images and abnormal images to make up a training data set, and inputting the training data set to the deep learning model so that the deep learning model can be trained to output a segmentation error heatmap image for the segmentation error region.

In the training, the deep learning model may be trained to compare the normal image and the abnormal image, and identify a segmentation error region based on a changed region in the abnormal image compared to the normal image.

The deep learning model may be provided as a single model to identify the segmentation error region from the preprocessed segmentation image upon the preprocessed segmentation image being provided, and apply a heatmap format to the segmentation error region.

The deep learning model may include: a first deep learning model configured to identify the segmentation error region from the preprocessed segmentation image upon the preprocessed segmentation image being provided; and a second deep learning model linked to the first deep learning model and configured to apply a heatmap format to the segmentation error region.

The calculation of the error risk may include calculating the error risk based on at least one of a maximum pixel value or a pixel value sum of the segmentation error heatmap image.

The provision of the auditing information may include: sorting the plurality of segmentation error heatmap images, in which the segmentation error risks have been calculated, in order of high segmentation error risk, and providing the input medical image, the output segmentation image, and the segmentation error heatmap image, in which the segmentation error risk is high, to the user.

Meanwhile, according to an embodiment of the disclosure, an apparatus for auditing of artificial intelligence-based medical image segmentation, the apparatus includes: a preprocessing module configured to generate a preprocessed segmentation image by receiving an input medical image and an output segmentation image provided from a medical image segmentation device and preprocessing the output segmentation image based on the input medical image; a heatmap generation module configured to generate a segmentation error heatmap image, which includes a segmentation error region in the preprocessed segmentation image, by inputting the preprocessed segmentation image provided from the preprocessing module to a deep learning model trained in advance; and a risk calculation module configured to calculate a segmentation error risk for the segmentation error region based on pixel values of the segmentation error heatmap image provided from the heatmap generation module, wherein auditing information for auditing accuracy of the output segmentation image based on the calculated segmentation error risk is provided to a user.

The preprocessing module may be configured to: receive the input medical image having a first data format and the output segmentation image having a second data format provided from the medical image segmentation device; and convert the output segmentation image having the second data format to have the same data format as the first data format of the input medical image.

The preprocessing module may be configured to: receive the input medical image and the output segmentation image, in which a segment region of the input medical image is subjected to a color overlay, provided from the medical image segmentation device; separate the output segmentation image into color components and gray components; and convert an image corresponding to the color components to have the same format as a format of the input medical image.

The heatmap generation module may be configured to: receive the input medical image and the segmentation image; and apply the preprocessed segmentation image to the deep learning model, which distinguishes between a normal image and an abnormal image, to output the segmentation error region in a heatmap format.

In training the deep learning model, a plurality of output segmentation images may be generated by inputting the plurality of input medical images to the medical image segmentation device, the plurality of output segmentation images may be divisionally labeled with normal images and abnormal images to make up a training data set, and the training data set may be input to the deep learning model so that the deep learning model can be trained to output a segmentation error heatmap image for the segmentation error region.

The deep learning model may be trained to compare the normal image and the abnormal image, and identify a segmentation error region based on a changed region in the abnormal image compared to the normal image.

The deep learning model may be provided as a single model to identify the segmentation error region from the preprocessed segmentation image upon the preprocessed segmentation image being provided, and apply a heatmap format to the segmentation error region.

The deep learning model may include: a first deep learning model configured to identify the segmentation error region from the preprocessed segmentation image upon the preprocessed segmentation image being provided; and a second deep learning model linked to the first deep learning model and configured to apply a heatmap format to the segmentation error region.

The risk calculation module may be configured to calculate the segmentation error risk based on at least one of a maximum pixel value or a pixel value sum of the segmentation error heatmap image.

In providing the auditing information, the plurality of segmentation error heatmap images, in which the segmentation error risks have been calculated, are sorted in order of high segmentation error risk, and the input medical image, the output segmentation image, and the segmentation error heatmap image, in which the segmentation error risk is high, may be provided to the user.

According to the disclosure, the apparatus and method for the auditing of the artificial intelligence-based medical image segmentation provide information necessary for the auditing to a user to determine whether the results output from the medical image segmentation device are accurate, thereby having an effect on ensuring the safe operations of the image segmentation technology.

The technical effects of the disclosure are not limited to the aforementioned effects, and other unmentioned technical effects may become apparent to those skilled in the art from the following description.

### BRIEF DESCRIPTION OF THE DRAWINGS

FIG. 1 is a conceptual view schematically showing an image segmentation auditing apparatus according to an embodiment,

FIG. 2 is a flowchart showing an auditing method using an image segmentation auditing apparatus according to an embodiment,

FIG. 3 is a conceptual view showing a preprocessing module of an image segmentation auditing apparatus according to an embodiment,

FIG. 4 is a conceptual view showing a first preprocessing method of an image segmentation auditing apparatus according to an embodiment,

FIG. 5 is a conceptual view showing a second preprocessing method of an image segmentation auditing apparatus according to an embodiment,

FIG. 6 is a conceptual view showing a method of generating a heatmap image in an auditing method using an image segmentation auditing apparatus according to an embodiment,

FIG. 7 is a conceptual view showing a method of training a deep learning model that generates a heatmap image in an auditing method using an image segmentation auditing apparatus according to an embodiment,

FIG. 8 is a conceptual view showing a method of calculating a segmentation risk based on a heatmap image in an auditing method using an image segmentation auditing apparatus according to an embodiment, and

FIG. 9 is a flowchart showing a method of providing auditing information in an image segmentation auditing apparatus according to an embodiment.

### DETAILED DESCRIPTION OF THE INVENTION

Hereinafter, embodiments of the disclosure will be described in detail with reference to the accompanying drawings. However, the disclosure is not limited to the embodiments set forth herein, but may be implemented in various different ways. The embodiments are provided to only complete the disclosure and allow those skilled in the art to understand the category of the disclosure. In the accompanying drawings, the shape, etc. of an element may be exaggerated for clear description, and like numerals refer to like elements.

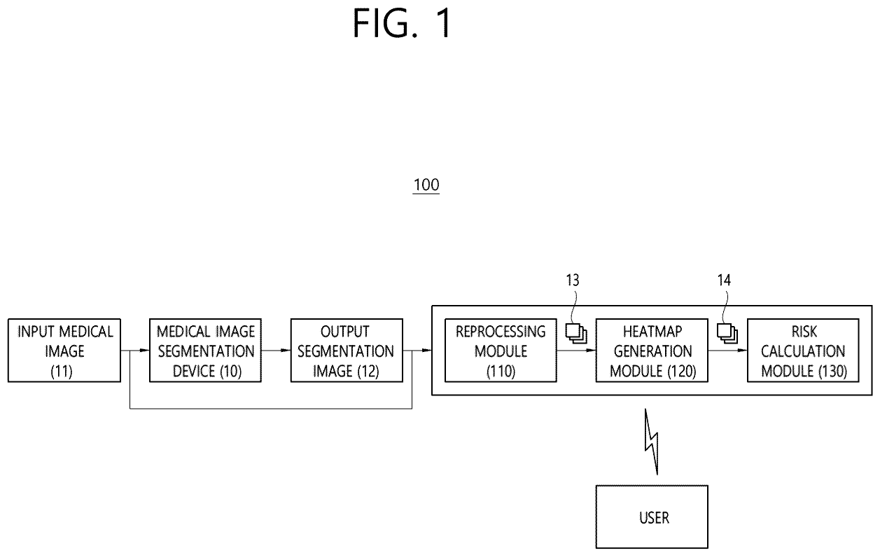

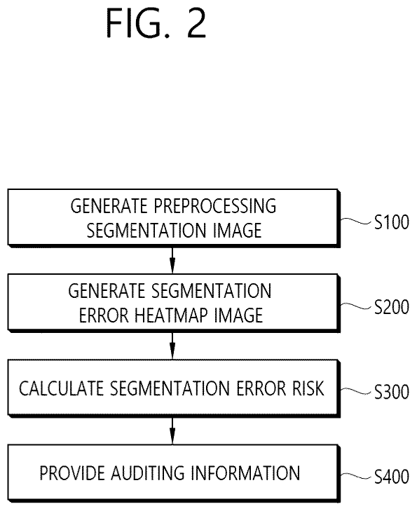

FIG. 1 is a conceptual view schematically showing an image segmentation auditing apparatus according to an embodiment, and FIG. 2 is a flowchart showing an auditing method using an image segmentation auditing apparatus according to an embodiment.

As shown in FIGS. 1 and 2, an image segmentation auditing apparatus 100 according to an embodiment outputs auditing information for auditing of an image segmentation processing process while interworking with a medical image segmentation device 10.

Here, the medical image segmentation device 10 may segment a medical image based on artificial intelligence. The medical image segmentation device 10 may apply an input medical image 11 provided from the outside to an artificial intelligence model, and output an output segmentation image 12 based on the artificial intelligence model.

Thus, the image segmentation auditing apparatus 100 allows a user to audit a medical image segmentation processing process, based on the input medical image 11 input to the medical image segmentation device 10 and the output segmentation image 12 output from the medical image segmentation device 10.

However, the output segmentation image 12 output from the medical image segmentation device 10 may be output in various forms depending on the artificial intelligence models applied to the medical image segmentation device 10.

For example, the artificial intelligence-based medical image segmentation device 10 may output the output segmentation image 12 by converting the data format of the input medical image 11. In other words, when the input medical image 11 of a first data format is provided, the medical image segmentation device 10 may input the input medical image 11 of the first data format to the artificial intelligence model, and output the output segmentation image 12, the data format of which is converted into the second data format by the artificial intelligence model. Here, the first data format and the second data format may be different in type, form, etc. from each other.

Alternatively, the artificial intelligence-based medical image segmentation device 10 may label a region of interest, i.e., a segment region on the input medical image 11 in various ways and output the output segmentation image 12 including the labeled segment region. In this case, when the input medical image 11 is provided, the medical image segmentation device 10 may input the input medical image 11 to the artificial intelligence model and output the output segmentation image 12, the segment reg1ion of which is changed in color. In other words, the medical image segmentation device 10 may output the output segmentation image 12 by performing a color overlay on the segment region of the input medical image 11 based on the artificial intelligence model. However, this is merely to describe an embodiment, and the segment region may be labeled on the input medical image 11 in various ways.

Meanwhile, both the input medical image 11 input to the medical image segmentation device 10 and the output segmentation image 12 output from the medical image segmentation device 10 may be transmitted to the image segmentation auditing apparatus 100. Thus, the image segmentation auditing apparatus 100 may include a preprocessing module 110 that preprocesses the output segmentation image 12 based on the input medical image 11 to generate a preprocessed segmentation image 13 (S100).

Then, the image segmentation auditing apparatus 100 generates a segmentation error heatmap image 14 (hereinafter referred to as a heatmap image) based on both the input medical image 11 and the preprocessed segmentation image 13 processed by the preprocessing module 110 in advance (S200). The image segmentation auditing apparatus 100 may include a heatmap generation module 120, and the heatmap generation module 120 may include a deep learning model trained in advance. Thus, the heatmap generation module 120 generates the heatmap image 14 by inputting the input medical image 11 and the preprocessed segmentation image 12 to the deep learning model trained in advance.

In addition, the image segmentation auditing apparatus 100 calculates a segmentation error risk based on the heatmap image 14 provided from the heatmap generation module 120 (S300). In this case, the image segmentation auditing apparatus 100 may include a risk calculation module 130, and the risk calculation module 130 may calculate the segmentation error risk based on a pixel value of the segmentation error heatmap image.

Finally, the image segmentation auditing apparatus 100 may provide an auditing result to a user based on the segmentation error risk provided from the risk calculation module 130 (S400).

Meanwhile, an auditing method using the image segmentation auditing apparatus will be described in detail with reference to the accompanying drawings. However, the foregoing elements will not be repetitively described, and assigned with the same reference numerals.

### <How to Perform Preprocessing>

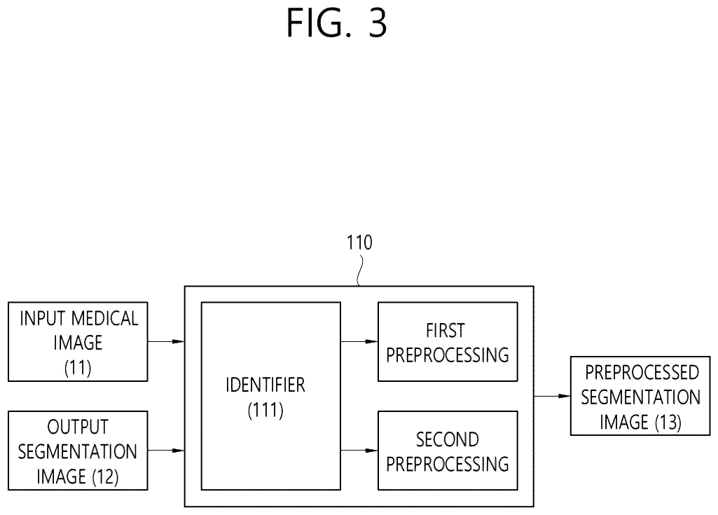

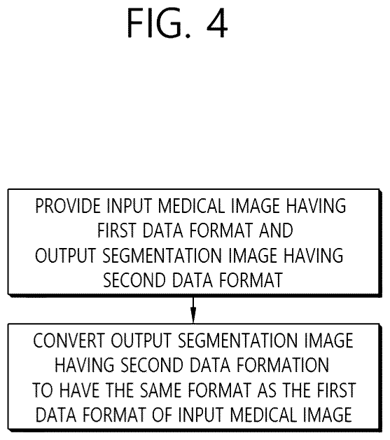

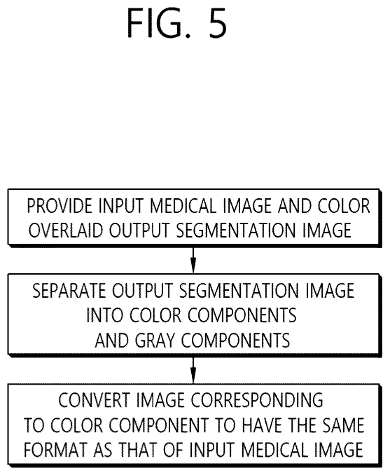

FIG. 3 is a conceptual view showing a preprocessing module of an image segmentation auditing apparatus according to an embodiment, and FIG. 4 is a conceptual view showing a first preprocessing method of an image segmentation auditing apparatus according to an embodiment. In addition, FIG. 5 is a conceptual view showing a second preprocessing method of an image segmentation auditing apparatus according to an embodiment.

As shown in FIGS. 3 to 5, the image segmentation auditing apparatus 100 according to an embodiment includes the preprocessing module 110.

The preprocessing module 110 may receive both the input medical image 11 and the output segmentation image 12. Thus, the preprocessing module 110 may preprocess the output segmentation image 12 based on the input medical image 11.

However, as described above, the artificial intelligence-based medical image segmentation device 10 may segment the medical image in various ways. Therefore, the preprocessing module 110 may identify a segmentation method based on the input medical image 11 and the output segmentation image 12 provided from the medical image segmentation device 10, and perform the preprocessing corresponding to the identified segmentation mode.

Thus, the preprocessing module 110 may include an identifier 111 that identifies the segmentation method when receiving the input medical image 11 and the output segmentation image 12. The identifier 111 may identify the segmentation method based on comparison between the input medical image 11 and the output segmentation image 12, and perform a first preprocessing method or a second preprocessing method according to the segmentation modes.

For example, when the input medical image 11 having the first data format and the output segmentation image 12 having the second data format are provided to the identifier 111, the identifier 111 may identify that the input medical image 11 and the output segmentation image 12 are different in data format from each other, thereby allowing the first preprocessing method to be performed.

By the first preprocessing method, when the input medical image 11 having the first data format and the output segmentation: image 12 having the second data format are provided, the second data format of the output segmentation image 12 having is converted into the same format as the first data format of the input medical image 11. In other words, the preprocessed segmentation image 13 may be generated by converting the output segmentation image 12 having the second data format to have the same format as the first data format of the input medical image 11. In other words, the preprocessed segmentation image 13 generated from the output segmentation image 12 may have data, of which the type, form, or the like format has been converted to be the same as that of the input medical image 11.

Alternatively, when the input medical image 11, and the output segmentation image 12 with the segment region labelled on the input medical image 11 are provided to the identifier 111, the identifier 111 may compare the input medical image 11 and the output segmentation image 12 to perform the second preprocessing method.

In particular, when the output segmentation image 12 is a color overlaid image, the second preprocessing method is performed to separate the color overlaid output segmentation image 12 into color components and gray components. Then, the image corresponding to the separated color components is converted to have the same format as that of the input medical image 11. In other words, the image of the color components is obtained by the second preprocessing method, and then subjected to the first preprocessing method, thereby generating the preprocessed segmentation image 13.

Then, the preprocessing module 110 provides the input medical image 11 and the preprocessed segmentation image 13 to the heatmap generation module 120.

### <How to Generate Segmentation Error Heatmap>

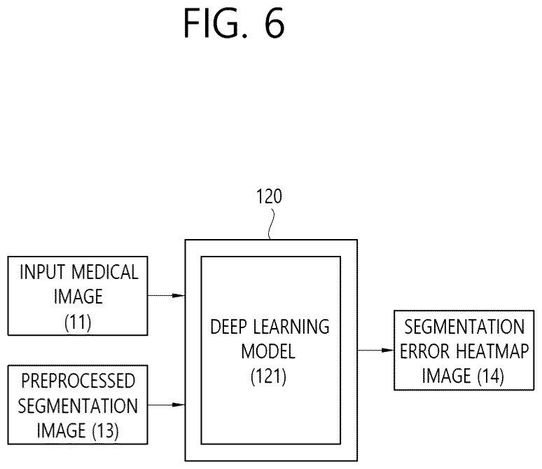

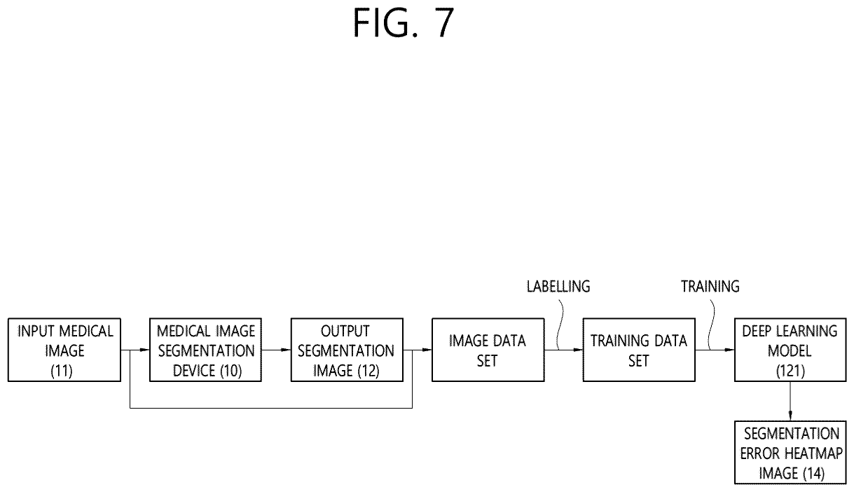

FIG. 6 is a conceptual view showing a method of generating a heatmap image in an auditing method using an image segmentation auditing apparatus according to an embodiment, and FIG. 7 is a conceptual view showing a method of training a deep learning model that generates a heatmap image in an auditing method using an image segmentation auditing apparatus according to an embodiment.

As shown in FIGS. 6 and 7, the image segmentation auditing apparatus 100 according to an embodiment includes the heatmap generation module 120.

The heatmap generation module 120 inputs the input medical image 11 and the preprocessed segmentation image 13 to a deep learning model 121 trained in advance, so that the deep learning model 121 can generate the heatmap image 14 in which a segmentation error region is expressed in a heatmap format. In other words, the heatmap generation module 120 may output the segmentation error region as visual graphics in the form of heat distribution. The deep learning model 121 may be trained in advance to generate a heatmap image selectively based on the segmentation error region.

For example, the deep learning model 121 is trained as follows. First, a large amount of medical image data sets is collected. Here, the medical image data sets may include, but not limited to, medical image data sets acquired from various medical apparatuses such as X-ray, CT and MRI apparatuses.

Then, in training the deep learning model 121, the medical image data sets are input as a plurality of input medical images 11 to the artificial intelligence-based medical image segmentation device 10, thereby generating a plurality of output segmentation images 12. Thus, an image data set for the input medical image 11 and the output segmentation image 12 is provided.

Then, in training the deep learning model 121, labeling is performed for a learning data set. In the labeling, the output segmentation images 12 are divisionally labeled with normal images and abnormal mages, and the labelled normal images and the labelled abnormal images make up a training data set.

Further, in training the deep learning model 121, the training data set is used to train the deep learning model 121. In this case, the normal image and the abnormal image are compared in training the deep learning model 121, and a changed region in the abnormal image compared to the normal image is identified as the segmentation error region. Then, the deep learning model 121 is trained to output a heatmap image 14 for the segmentation error region.

The deep learning model 121 may be provided as a single model to identify a segmentation error region and generate a heatmap for the identified segmentation error region. Alternatively, the deep learning model 121 may operate to link a plurality of models together so that a first deep learning model can identify the segmentation error region and a second deep learning model can generate the heatmap for the identified segmentation error region.

Thus, when the input medical image 11 and the preprocessed segmentation image 13 are provided by the preprocessing module 110, the heatmap generation module 120 may input the input medical image 11 and the preprocessed segmentation image 13 to the deep learning model 121 trained in advance, and generate the heatmap image 14. Then, the heatmap generation module 120 may provide the generated heatmap image 14 to the risk calculation module 130.

### <How to Calculate Error Risk and Provide Auditing Information>

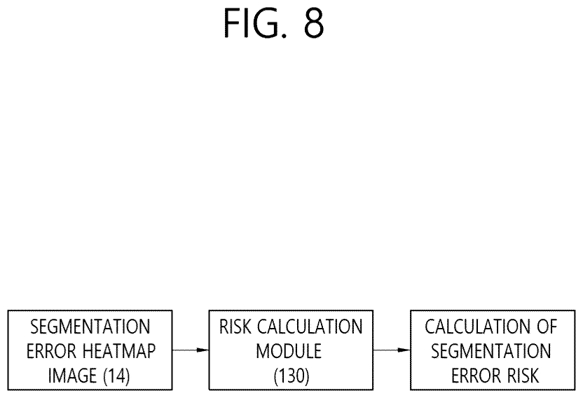

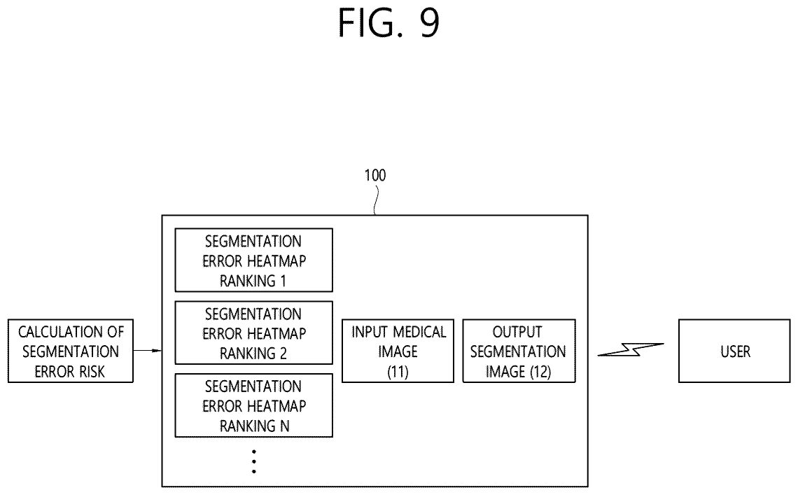

FIG. 8 is a conceptual view showing a method of calculating a segmentation risk based on a heatmap image in an auditing method using an image segmentation auditing apparatus according to an embodiment, and FIG. 9 is a flowchart showing a method of providing auditing information in an image segmentation auditing apparatus according to an embodiment.

As shown in FIGS. 8 and 9, the image segmentation auditing apparatus 100 according to an embodiment includes the risk calculation module 130. The risk calculation module 130 may calculate a risk of the segmentation error for the heatmap image 14.

As described above, the heatmap refers to graphics in which values of data are converted into colors and visualized in the form of heat distribution. Thus, in the heatmap image 14 output from the deep learning model 121, the segmentation error region in the preprocessed segmentation image 13 may be expressed in the form of heat distribution. For example, a region with a high probability of segmentation error may be expressed in red, and a region with a moderate probability of segmentation error may be expressed in green. In addition, a region with a low probability of segmentation error may be expressed in blue.

Thus, the risk calculation module 130 may calculate the segmentation error risk by using the maximum pixel value or the pixel value sum based on the colors in the heatmap image 14 provided from the heatmap generation module 120.

Then, the image segmentation auditing apparatus 100 sorts the plurality of heatmap images, in which the segmentation error risks have been calculated, so as to provide the auditing information to a user. In this case, the image segmentation auditing apparatus 100 may sort the heatmap images, which have been provided by the risk calculation module 130, in order of high segmentation error risk.

Further, the image segmentation auditing apparatus 100 provides the input medical image 11, the output segmentation image 12, and the heatmap image 14 to a user by a preset communication method. Thus, a user compares the input medical image 11, the output segmentation image 12, and the heatmap image 14, thereby auditing the accuracy of the segmentation result in the image segmentation processing process of the medical image segmentation device 10.

Thus, the apparatus and method for the auditing of the artificial intelligence-based medical image segmentation provide information necessary for the auditing to a user to determine whether the results output from the medical image segmentation device are accurate, thereby ensuring the safe operations of the image segmentation technology.

Although a few embodiments of the disclosure have been described above and illustrated in the accompanying drawings, the embodiments should not be construed as limiting the technical spirit of the disclosure. The scope of the disclosure is limited only by the subject matters disclosed in the appended claims, and the technical spirit of the disclosure may be modified and changed in various forms by a person having ordinary knowledge in the art. Therefore, such modification and change obvious to those skilled in the art will fall within the scope of the disclosure.

What is claimed is:

1. A method of auditing of artificial intelligence-based medical image segmentation, the method comprising:
performing preprocessing to generate a preprocessed segmentation image by receiving an input medical image and an output segmentation image provided from a medical image segmentation device and preprocessing the output segmentation image based on the input medical image;
generating a heatmap image to generate a segmentation error heatmap image, which comprises a segmentation error region in the preprocessed segmentation image, by inputting the preprocessed segmentation image to a deep learning model trained in advance;
calculating an error risk to calculate a segmentation error risk for the segmentation error region based on pixel values of the segmentation error heatmap image; and
providing auditing information to provide the auditing information for auditing accuracy of the output segmentation image based on the calculated segmentation error risk to a user.

2. The method of claim 1, wherein the performance of the preprocessing comprises:
receiving the input medical image having a first data format and the output segmentation image having a second data format provided from the medical image segmentation device; and
converting the output segmentation image having the second data format to have the same data format as the first data format of the input medical image.

3. The method of claim 1, wherein the performance of the preprocessing comprises:
receiving the input medical image and the output segmentation image, in which a segment region of the input medical image is subjected to a color overlay, provided from the medical image segmentation device;
separating the output segmentation image into color components and gray components; and
converting an image corresponding to the color components to have the same format as a format of the input medical image.

4. The method of claim 1, wherein the generation of the heatmap image comprises:
receiving the preprocessed segmentation image; and
applying the preprocessed segmentation image to the deep learning model, which distinguishes between a normal image and an abnormal image, to output the segmentation error region in a heatmap format.

5. The method of claim 4, further comprising: in training the deep learning model,
generating a plurality of output segmentation images by inputting the plurality of input medical images to the medical image segmentation device,
labeling the plurality of output segmentation images divisionally with normal images and abnormal images to make up a training data set, and
inputting the training data set to the deep learning model so that the deep learning model can be trained to output a segmentation error heatmap image for the segmentation error region.

6. The method of claim 5, wherein, in the training, the deep learning model is trained to compare the normal image and the abnormal image, and identify a segmentation error region based on a changed region in the abnormal image compared to the normal image.

7. The method of claim 6, wherein the deep learning model is provided as a single model to identify the segmentation error region from the preprocessed segmentation image upon the preprocessed segmentation image being provided, and apply a heatmap format to the segmentation error region.

8. The method of claim 7, wherein the deep learning model comprises:
a first deep learning model configured to identify the segmentation error region from the preprocessed segmentation image upon the preprocessed segmentation image being provided; and
a second deep learning model linked to the first deep learning model and configured to apply a heatmap format to the segmentation error region.

9. The method of claim 1, wherein the calculation of the error risk comprises calculating the error risk based on at least one of a maximum pixel value or a pixel value sum of the segmentation error heatmap image.

10. The method of claim 1, wherein the provision of the auditing information comprises:
sorting the plurality of segmentation error heatmap images, in which the segmentation error risks have been calculated, in order of high segmentation error risk, and
providing the input medical image, the output segmentation image, and the segmentation error heatmap image, in which the segmentation error risk is high, to the user.

11. An apparatus for auditing of artificial intelligence-based medical image segmentation, the apparatus comprising:
a preprocessing module configured to generate a preprocessed segmentation image by receiving an input medical image and an output segmentation image provided from a medical image segmentation device and preprocessing the output segmentation image based on the input medical image;
a heatmap generation module configured to generate a segmentation error heatmap image, which comprises a segmentation error region in the preprocessed segmentation image, by inputting the preprocessed segmentation image provided from the preprocessing module to a deep learning model trained in advance; and
a risk calculation module configured to calculate a segmentation error risk for the segmentation error region based on pixel values of the segmentation error heatmap image provided from the heatmap generation module,
wherein auditing information for auditing accuracy of the output segmentation image based on the calculated segmentation error risk is provided to a user.

12. The apparatus of claim 11, wherein the preprocessing module is configured to:
receive the input medical image having a first data format and the output segmentation image having a second data format provided from the medical image segmentation device; and
convert the output segmentation image having the second data format to have the same data format as the first data format of the input medical image.

13. The apparatus of claim 11, wherein the preprocessing module is configured to:
receive the input medical image and the output segmentation image, in which a segment region of the input medical image is subjected to a color overlay, provided from the medical image segmentation device;
separate the output segmentation image into color components and gray components; and
convert an image corresponding to the color components to have the same format as a format of the input medical image.

14. The apparatus of claim 1, wherein the heatmap generation module is configured to:
receive the segmentation image; and
apply the preprocessed segmentation image to the deep learning model, which distinguishes between a normal image and an abnormal image, to output the segmentation error region in a heatmap format.

15. The apparatus of claim 14, wherein, in training the deep learning model,
a plurality of output segmentation images is generated by inputting the plurality of input medical images to the medical image segmentation device,
the plurality of output segmentation images are divisionally labeled with normal images and abnormal images to make up a training data set, and
the training data set is input to the deep learning model so that the deep learning model can be trained to output a segmentation error heatmap image for the segmentation error region.

16. The apparatus of claim 15, wherein the deep learning model is trained to compare the normal image and the abnormal image, and identify a segmentation error region based on a changed region in the abnormal image compared to the normal image.

17. The apparatus of claim 16, wherein the deep learning model is provided as a single model to identify the segmentation error region from the preprocessed segmentation image upon the preprocessed segmentation image being provided, and apply a heatmap format to the segmentation error region.

18. The apparatus of claim 17, wherein the deep learning model comprises:
a first deep learning model configured to identify the segmentation error region from the preprocessed segmentation image upon the preprocessed segmentation image being provided; and
a second deep learning model linked to the first deep learning model and configured to apply a heatmap format to the segmentation error region.

19. The apparatus of claim 11, wherein the risk calculation module is configured to calculate the segmentation error risk based on at least one of a maximum pixel value or a pixel value sum of the segmentation error heatmap image.

20. The apparatus of claim 11, wherein, in providing the auditing information,
the plurality of segmentation error heatmap images, in which the segmentation error risks have been calculated, are sorted in order of high segmentation error risk, and
the input medical image, the output segmentation image, and the segmentation error heatmap image, in which the segmentation error risk is high, are provided to the user.
